(19) United States
(12) Patent Application Publication
(10) Pub. No.: US 2006/0121124 A1
(43) Pub. Date: Jun. 8, 2006

(54) Method of using cartilage extract for increasing blood parameters

(75) Inventors: Eric Dupont, Ste Petronille (CA); Jocelyn Berube, L'Ancienne-Lorette (CA)

Correspondence Address: OBLON, SPIVAK, MCCLELLAND, MAIER & NEUSTADT, P.C., 1940 DUKE STREET, ALEXANDRIA, VA, 22314, US

(73) Assignee: AETERNA ZENTARIS INC., Quebec (CA)

(21) Appl. No.: 11/271,846

(22) Filed: Nov. 14, 2005

Related U.S. Application Data
(60) Provisional application No. 60/627,945, filed on Nov. 16, 2004.

(51) Int. Cl. A61K 35/34

(52) U.S. Cl. 424/548

(57) ABSTRACT

A method of increasing at least one blood parameter in a mammal by administering cartilage extract to said mammal, with the proviso that when said at least one blood parameter is low prior to administering said cartilage extract, said mammal is not co-administered an anticancer agent; compositions containing said cartilage extract and uses thereof.

### REFERENCE TO RELATED APPLICATIONS

This application claims priority to U.S. provisional application No. 60/627,945 filed Nov. 16, 2004, incorporated herein by reference.

### FIELD OF THE INVENTION

The present invention relates to a method of using cartilage extract for increasing blood parameters. More specifically, the present invention is concerned with using cartilage extract for increasing erythrocyte count, hematocrit and hemoglobin in mammals and more particularly humans.

### BACKGROUND OF THE INVENTION

Cartilage extracts and particularly shark cartilage extract are known to inhibit the angiogenic process which is known to sustain cancer cell growth and facilitate their dissemination. See for instance U.S. Pat. No. 5,618,925, U.S. Pat. No. 5,985,839; U.S. Pat. No. 6,025,334; U.S. Pat. No. 6,028,118, U.S. Pat. Nos. 6,635,285; 6,380,366, 6,168,807 and 6,506,414.

Cartilage extract is also known to decrease and/or prevent certain toxic side effects caused by chemotherapy. See for instance U.S. Pat. No. 6,383,522. It is well admitted that every chemotherapeutic regimen will have some deleterious side effects on normal tissues, the most common being myelosuppression, (manifested as anemia, leucopenia and thrombocytopenia) nausea and vomiting, stomatitis and alopecia (Harrison's Principles of Internal Medicine). Usually, the total number of white blood cells in the circulating blood reach its lowest point (termed “nadir”) 14 days following treatment, with a gradual return to normal at Day 28. This decrease in white blood cells limits the frequency of treatments to once every 21 to 28 days (some treatments needing a delay of 6 weeks). U.S. patent application Ser. No. 10/087,041 showed that in the Lewis Lung carcinoma (LLC) mice model shark cartilage extract was able protect against certain chemotherapeutic agents (specifically cisplatin) deleterious side effects, namely weight loss and peripheral white blood cells decrease. Shark cartilage extract alone however was not able to increase body weight or white blood cell counts in these mice models.

Furthermore, erythrocyte count, hematocrit and hemoglobin were shown to be decreased in animals treated with cisplatin alone, but remained within normal levels in animals treated with cisplatin and shark cartilage extract in combination (Evans et al., 2001). Cartilage extract was never shown however to increase erythrocyte cell count, hemoglobin or hematocrit levels in mammals not undergoing chemotherapy.

There are many diseases and conditions that cause these blood parameters to decrease including anemia associated with endocrine disorders such as Addison's disease and hyperthyroidism/hypothyroidism; anemia associated with bone marrow diseases such as a plastic anemia and myelodysplasia; anemia of aging; anemia of chronic disease such as ankylosing spondylitis, arteritis giant cell, cancer, diabetes, inflammatory bowel disease such as Crohn's disease and ulcerative colitis, kidney diseases such as chronic renal failure and glomerulonephritis, lung abscess, mixed connective tissue, rheumatoid arthritis, sarcoidosis, scleroderma, subacute endocarditis, systemic lupus erythematosus, tecidual injury (fracture), uremic syndrome, vasculitis; anemia of infection such as hepatitis, HIV/AIDS, malaria and tuberculosis; diamond-blackfan anemia; drepanocytic anemia; fanconi's anemia; hemolytic anemia; hypoplastic anemia; iron deficiency anemia; myelophthisic anemia; pancytopenia; pernicious anemia; porphyria; sickle cell anemia; sideroblastic anemia; and thalassemia.

TABLE 1
Prevalence of anemia in certain types of cancer

| | Prevalence of |
|---|---|
| Type of cancer | anemia (%) |
| Multiple myeloma | 62-90 |
| Hodgkin disease | 32-86 |
| Cervical | 67-82 |
| Mixed type tumors | 36-75 |
| Colorectal | 50-67 |
| Non-Hodgkin lymphoma | 32-66 |
| Head/Neck | 29-64 |
| Lung/Bronchus | 12-63 |
| Breast | 41-44 |
| Kidney | 39 |
| Ovarian | 26 |
| Prostate | 5-32 |

Source: National Anemia Action Council: Amgen anemia systematic review, 2000.

Blood parameters within normal range are good indicators of quality of life and should help a patient get through its therapy with more energy, optimism and, most of all, more chances of long term'survival. It may also improve the efficacy of chemotherapeutic and radiotherapeutic treatments, which depends on proper tissue oxygenation for maximal efficacy. Furthermore, it decreases the overall pro-angiogenic pressure by increasing oxygenation of tissues and organs, thereby decreasing the release of pro-angiogenic factors such as VEGF (Dunst et al., 2002).

It may also be desirable for individuals with normal blood parameters to increase these parameters and thus increase oxygen transport and consequently athletic performance or energy.

There remains a need for an agent able to increase or normalize blood parameters in individuals including those suffering from low blood parameters and increase blood parameters in those having normal blood parameters.

The present invention seeks to meet any of these needs.

The present description refers to a number of documents, the content of which is herein incorporated by reference in their entirety.

### SUMMARY OF THE INVENTION

It was discovered that cartilage extract increases blood parameters in humans.

The applicant has shown that cartilage extract is able to normalize or increase blood parameters in a human clinical trial following post-hoc exploratory analyses. Late stage renal cell carcinoma (RCC) patients showed signs of hematological abnormalities (tendency to anemia) since hematocrit was 16% lower than normal values in the cohort. This parameter remained relatively stable during the course of the study, showing neither improvement nor deterioration in the placebo group. However shark cartilage extract (SCE) exhibited a significant effect on both erythrocyte counts and hematocrit. It is also reasonably predictable that shark cartilage extract will be able to normalize or increase blood parameters in individuals suffering from other diseases which cause anemia including anemia associated with endocrine disorders such as Addison's disease and hyperthyroidism/hypothyroidism; anemia associated with bone marrow diseases such as aplastic anemia and myelodysplasia; anemia of aging; anemia of chronic disease such as ankylosing spondylitis, arteritis giant cell, cancer such as those described in Table 1, diabetes, inflammatory bowel disease such as Crohn's disease and ulcerative colitis, kidney diseases such as chronic renal failure and glomerulonephritis, lung abscess, mixed connective tissue, rheumatoid arthritis, sarcoidosis, scleroderma, subacute endocarditis, systemic lupus erythematosus, tecidual injury (fracture), uremic syndrome, vasculitis; anemia of infection such as hepatitis, HIV/AIDS, malaria and tuberculosis; diamond-blackfan anemia; drepanocytic anemia; fanconi's anemia; hemolytic anemia; hypoplastic anemia; iron deficiency anemia; myelophthisic anemia; pancytopenia; pernicious anemia; porphyria; sickle cell anemia; sideroblastic anemia; thalassemia; treatment-induced anemia such as AZT, and chemotherapy.

It has further been discovered that SCE is able to increase blood parameters in healthy individuals. Hence, the extract of the present invention may be used to enhance athletic performance.

There is a direct link between hemoglobin concentrations in blood and the ability to use oxygen, as measured by VO2max. When hemoglobin concentration is higher, the blood can carry more oxygen. This builds up a functional reserve that pushes forward the limits of the body. Prior studies have shown that each increment of 1 g/dL in hemoglobin increases physical performances by 1-4% (Warren G L and Cureton K J, 1989). The profit lays in the increase in fitness, energy, and endurance that come along with better oxygen utilization. Any individual may benefit from this increase in oxygen transport although untrained individual may feel more dramatic benefits then super athletes already near the upper limits of their capacities.

This invention relates to uses or method of using a cartilage extracts in all forms lyophilizate or solid extract, a liquid cartilage extract and to liquid fractions thereof.

As used herein, the terminology “blood parameters” is meant to refer to total erythrocyte number in a given volume, hematocrit or hemoglobin concentration in peripheral blood, (dimension: g/L or mol/L) and any combination thereof.

As used herein, the term “hematocrit” refers to the ratio of the volume of blood cells on the total volume of plasma. It is made by centrifugation of an unfractionated blood sample using a tabletop centrifuge to obtain a pellet of packed blood cells, of which erythrocyte is the major component.

As used herein, the terminology “normal blood parameter” refers to what is considered normal in recognized textbooks for the sub-group to which the treated subject belong. Without being so limited, this terminology generally refers herein to values within the range presented in Table 2 below for instance. Consequently, “normalizing blood parameters” means herein increasing the blood parameters in individuals suffering from anemia up to values within the normal range of individuals not suffering from anemia as presented in Table 2 below for instance. Any statistically significant “increase” of a blood parameter in a treated subject as compared to its blood parameter prior to the treatment is encompassed within the scope of the present invention. Hence, the present application encompasses cases where the increase in a blood parameter may not be sufficient to bring this blood parameter within the “normal” range as defined herein but is nevertheless statistically significant, and cases where the baseline blood parameter (that before treatment) of the subject is already within “normal ranges” as defined herein but wherein the increase achieved by the cartilage extract of the present invention is statistically significant.

As used herein the term “anemia” is meant to refer to low erythrocytes count, and/or low hemoglobin. Without being so limited, WHO defines anemia as hemoglobin level below 13 g/dL in men and 12 g/dL in women, or hematocrit under 39% in men and 36% in women, although different values are accepted (See http://www.who.int/nut/documents/ida_assessment_prevention_control.pdf at page 49). Anemia is not necessarily linked to leucopenia. A complete myelossuppression (destruction of bone marrow) will likely lead to a lowering in both parameters, but changes in factors that are specific to the erythrocyte lineage is likely to induce only anemia, without affecting white blood cells (example: iron deficiency). Also the ability of a compound to increase erythrocyte count is not predictive of the ability of the compound to increase the white blood cells concentration White blood cells and erythrocytes follow a distinctive path of maturation and involve a totally different set of cytokines and growth factors in their maturation. Erythropoietin (EPO) is a growth factor specific to the maturation of erythrocytes. It is still unclear whether the stimulation of erythrocytes in the tested population arose from a change in inflammatory cytokines, increase in EPO sensitivity, increase in EPO synthesis, presence of endogenous EPO in the cartilage extract, reduced erythrocytes destruction, improved iron utilization or any other biological pathway.

TABLE 2
normal ranges of blood parameters for men and women

| Parameter | Normal range for men | Normal range for women |
|---|---|---|
| Erythrocytes | 4.6-5.4 | 4.0-4.8 |
| (×10¹² cells/L) | | |
| Hematocrit (%) | 41-47 | 37.5-43.5 |
| Hemoglobin (g/dL) | 14.0-16.0 | 12.5-15.0 |

Source: Clinical Laboratory Testings, Ed. McGraw-Hill, 2001

As used herein, the terminology “low blood parameter” is meant to refer to a level of blood parameter that is lower than normal blood parameter for the treated subject, or if not known, lower than recognized normal blood parameters for the subpopulation to which the treated subject belongs.

As used herein the term “SCE” is a contraction of shark cartilage extract.

Examples of processes for obtaining cartilage extract to be used for the purpose described herein are described in U.S. Pat. No. 5,618,925, U.S. Pat. No. 5,985,839; U.S. Pat. No. 6,025,334; U.S. Pat. No. 6,028,118, WO 0004910 and WO 02/062359.

Pharmaceutical compositions of the present invention can be administered orally, nasally, intravenously, intramuscularly, subcutaneously, sublingually, intrathecally, or intradermally. The route of administration can depend on a variety of factors, such as the environment (e.g., the circumstances resulting in low blood parameters or desirability of increasing blood parameters when the patient has normal blood parameters) and therapeutic goals. As used herein, mammals generally refer to humans, but also can include domesticated mammals (e.g., dogs, cats, and livestock such as cows, horses, pigs, and sheep) in which increase of at least one blood parameter is desirable.

Dosage

Any amount of a pharmaceutical and/or nutraceutical and/or dietary supplement compositions can be administered to a mammal. The dosages will depend on many factors including the mode of administration. Typically, the amount of cartilage extract contained within a single dose will be an amount that effectively increases at least one blood parameter.

In some embodiments, the effective amount of the cartilage extract composition can be altered. Useful effective amount concentrations include amounts ranging from about 0.01% to about 10% of a total diet on a weight by weight basis, from about 1% to about 6% of a total diet on a weight by weight basis, or from about 2% to about 6% of a total diet on a weight by weight basis. Preferably, the amount of SCE able to induce an increase in blood parameters in said mammals range from 5 ml to 240 ml per day based on oral administration, providing that said mammal is a human.

By way of example, pharmaceutical and/or nutraceutical and/or dietary supplement composition of the invention can be in the form of a liquid, solution, suspension, pill, capsule, tablet, gelcap, powder, gel, ointment, cream, nebulae, mist, atomized vapor, aerosol, or phytosome. For oral administration, tablets or capsules can be prepared by conventional means with pharmaceutically acceptable excipients such as binding agents, fillers, lubricants, disintegrants, or wetting agents. The tablets can be coated by methods known in the art. Liquid preparations for oral administration can take the form of, for example, solutions, syrups, or suspension, or they can be presented as a dry product for constitution with saline or other suitable liquid vehicle before use. Pharmaceutical and/or nutraceutical and/or dietary supplement composition of the invention also can contain pharmaceutically acceptable additives such as suspending agents, emulsifying agents, non-aqueous vehicles, preservatives, buffer salts, flavoring, coloring, and sweetening agents as appropriate. Preparations for oral administration also can be suitably formulated to give controlled release of the active ingredients.

In addition, pharmaceutical and/or nutraceutical and/or dietary supplement composition of the invention can contain a pharmaceutically acceptable carrier for administration to a mammal, including, without limitation, sterile aqueous, or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water, alcohol, saline, and buffered solutions. Pharmaceutically acceptable carriers also can include physiologically acceptable aqueous vehicles (e.g., physiological saline) or other known carriers appropriate to specific routes of administration.

Cartilage extract may be incorporated into dosage forms in conjunction with any of the vehicles which are commonly employed in pharmaceutical preparations, e.g. talc, gum arabic, lactose, starch, magnesium stearate, cocoa butter, aqueous or non-aqueous solvents, oils, paraffin derivatives or glycols. Emulsions such as those described in U.S. Pat. No. 5,434,183, may also be used in which vegetable oil (e.g., soybean oil or safflower oil), emulsifying agent (e.g., egg yolk phospholipid) and water are combined with glycerol. Methods for preparing appropriate formulations are well known in the art (see e.g., Remington's Pharmaceutical Sciences, 16th Ed., 1980, A. Oslo Ed., Easton, Pa.).

In cases where parenteral administration is elected as the route of administration, preparations containing cartilage extract may be provided to patients in combination with pharmaceutically acceptable sterile aqueous or non-aqueous solvents, suspensions or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. Intravenous vehicles may include fluid and nutrient replenishers, electrolyte replenishers, such as those based upon Ringer's dextrose, and the like.

These are simply guidelines since the actual dose must be carefully selected and titrated by the attending physician based upon clinical factors unique to each patient or by a nutritionist. The optimal daily dose will be determined by methods known in the art and will be influenced by factors such as the age of the patient and other clinically relevant factors. In addition, patients may be taking medications for other diseases or conditions. The other medications may be continued during the time that cartilage extract is given to the patient, but it is particularly advisable in such cases to begin with low doses to determine if adverse side effects are experienced.

Any route of administration or dosage form disclosed in any of U.S. Pat. No. 5,618,925, U.S. Pat. No. 5,985,839; U.S. Pat. No. 6,025,334; U.S. Pat. No. 6,028,118, U.S. Pat. Nos. 6,635,285; 6,380,366, 6,168,807, U.S. Pat. Nos. 6,383,522 and 6,506,414 may be used for the purpose described herein.

More specifically, in accordance with the present invention, there is provided a method of increasing at least one blood parameter in a mammal comprising administering cartilage extract to said mammal, with the proviso that when said at least one blood parameter is low prior to administering said cartilage extract, said mammal is not co-administered an anticancer agent. In a specific embodiment, the at least one blood parameter is erythrocyte cell count/total erythrocyte number in a given volume. In an other specific embodiment, the at least one blood parameter is hematocrit level. In an other specific embodiment, the at least one blood parameter is haemoglobin level/concentration. In an other specific embodiment, said cartilage extract is shark cartilage extract. In an other specific embodiment, said mammal is a human. In an other specific embodiment, said at least one blood parameter is low in said mammal prior to administering said cartilage extract. In an other specific embodiment, said at least one blood parameter is normal in said mammal prior to administering said cartilage extract.

According to an other aspect of the present invention, there is also provided a use of cartilage extract for increasing at least one blood parameter in a mammal comprising administering cartilage extract to said mammal, with the proviso that when said at least one blood parameter is low prior to administering said cartilage extract, said mammal is not co-administered an anticancer agent.

According to an other aspect of the present invention, there is also provided a use of cartilage extract for the preparation of a medicament for increasing at least one blood parameter in a mammal comprising administering cartilage extract to said mammal, with the proviso that when said at least one blood parameter is low prior to administering said cartilage extract, said mammal is not co-administered an anticancer agent.

In specific embodiments of the uses according to the present invention, the at least one blood parameter is erythrocyte cell count/total erythrocyte number in a given volume. In an other specific embodiment, the at least one blood parameter is hematocrit level. In an other specific embodiment, the at least one blood parameter is haemoglobin level/concentration. In an other specific embodiment, said cartilage extract is shark cartilage extract. In an other specific embodiment, said mammal is a human. In an other specific embodiment, said at least one blood parameter is low in said mammal prior to administering said cartilage extract. In an other specific embodiment, said at least one blood parameter is normal in said mammal prior to administering said cartilage extract.

According to an other aspect of the present invention, there is also provided a pharmaceutical and/or nutraceutical and/or dietary composition containing a cartilage extract for increasing at least one blood parameter in a mammal and if appropriate customary pharmaceutically acceptable auxiliaries, additives and/or carriers with the proviso that when said at least one blood parameter is low prior to administration of said pharmaceutical composition, said mammal is not co-administered an anticancer agent. In an other specific embodiment, the at least one blood parameter is erythrocyte cell count/total erythrocyte number in a given volume. In an other specific embodiment, the at least one blood parameter is hematocrit level. In an other specific embodiment, the at least one blood parameter is haemoglobin level/concentration. In an other specific embodiment, said cartilage extract is shark cartilage extract. In an other specific embodiment, said mammal is a human. In an other specific embodiment, said at least one blood parameter is low in said mammal prior to administering said cartilage extract. In an other specific embodiment, said at least one blood parameter is normal in said mammal prior to administering said cartilage extract.

According to other specific embodiments of the present invention, there is provided methods, use and compositions as described herein wherein said mammal and more preferably said human has cancer. In a further specific embodiment, said cancer is late stage renal cell carcinoma.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1 presents the mean erythrocytes count of all patients in the study. Mean plateau values are presented as mean ± Standard Error of the Mean (sem) and represents the mean of all available data from weeks 24 to 60;

FIG. 2 presents the mean hematocrit values of all patients in the study. Mean plateau values are presented as mean ± sem and represents the mean of all available data from weeks 24 to 60;

FIG. 3 presents the mean erythrocyte count of healthy humans administered shark cartilage extract. Data are presented as mean±sem;

FIG. 4 presents the mean hematocrit of healthy humans administered shark cartilage extract. Data are presented as mean±sem; and

FIG. 5 presents the mean haemoglobin level of healthy humans administered shark cartilage extract. Data are presented as mean±sem.

### DESCRIPTION OF ILLUSTRATIVE EMBODIMENTS

The present invention is illustrated in further details by the following non-limiting examples.

### EXAMPLE 1

### Obtention of Shark Cartilage Extract

The shark cartilage extract was obtained as described in U.S. Pat. No. 6,635,285 which is herein incorporated by reference using a Mustelus schmitti shark species.

### EXAMPLE 2

### Administration of Shark Cartilage Extract to Human Patients with Late Stage Refractory Renal Cell Carcinoma

Shark cartilage extract was obtained as described in Example 1.

A prospective, multi-centered, randomized, double-blinded, placebo-controlled trial for measuring the efficacy of 120 ml bid of shark cartilage extract in patients with refractory renal cell carcinoma (RCC) as monotherapy was conducted.

Shark cartilage extract was obtained as described in Example 1. Enrolment criteria includes histologically-confirmed renal cell adenocarcinoma, disease progression within 16 weeks following first line therapy (IL-2 and/or IFN), unresectable metastatic disease, no more than one round of prior anticancer treatment, namely interferon and IL-2 therapy, and absence of brain metastases.

The endpoint of the study was efficacy, which was measured by assessing overall survival time as a primary endpoint. Secondary efficacy markers included time to progression and one-year survival rate. Overall tumor response rate, duration of response and quality of life were assessed as exploratory parameters.

Patients were having scheduled visits with their oncologist: every 4 weeks until week 24 following randomization, and every 12 weeks thereafter. During these visits, blood samples were drawn and were used for determination of biochemical serum characterisation, along with cell counts.

The results of the study were published on September 2003. The efficacy endpoints of the study were not met, although a significant increase in survival time was observed in a subpopulation of 21 shark cartilage extract patients (compared to 17 placebo patients) following an exploratory post-hoc analysis.

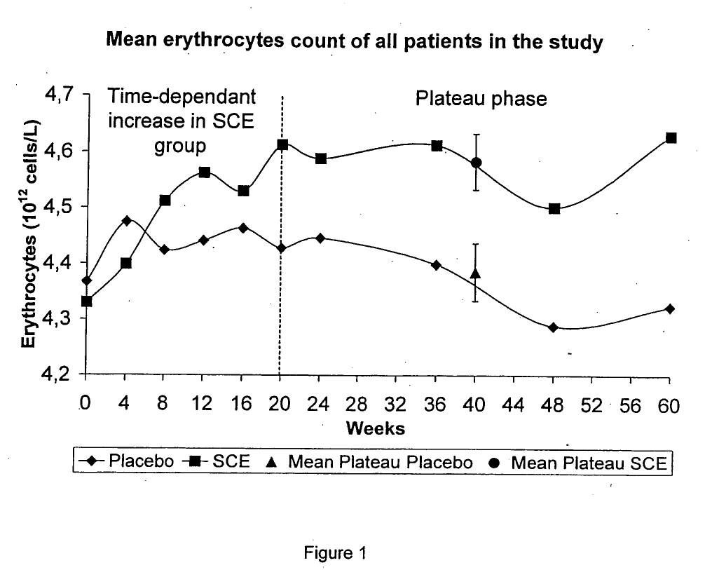

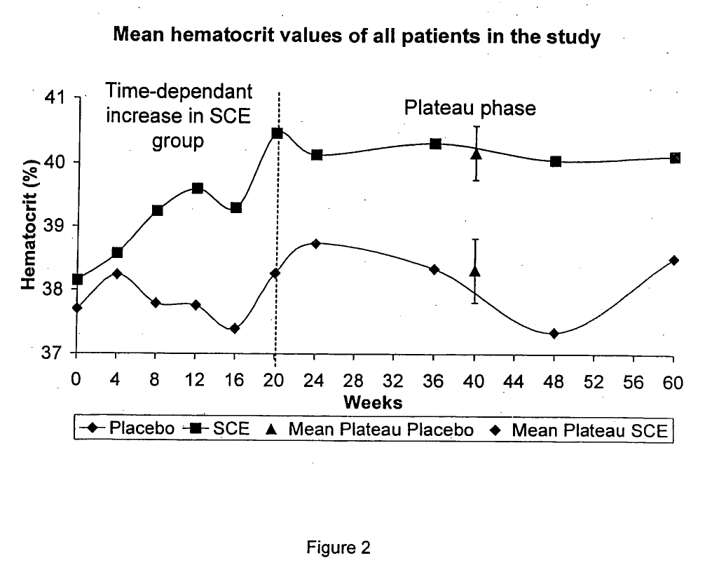

A retrospective analysis of blood parameters conducted on all available data (i.e. from 297 patients for baseline lab data; from 147 patients at week 20; and from 52 patients at week 60) showed that some parameters were significantly improved by the shark cartilage extract compared to placebo. These results were not known at the time of the initial disclosure of the efficacy results and have not been disclosed since. These results are summarized in FIGS. 1 and 2.

This RCC cohort of late stage patients showed signs of hematological abnormalities (tendency to anemia) were seen since hematocrit was 16% lower than normal values. This parameter remained relatively stable during the course of the study, showing neither improvement nor deterioration in the placebo group. However Shark cartilage extract exhibited a significant effect on both erythrocytes count and hematocrit (FIGS. 1 and 2). Hence, patients receiving the shark cartilage extract had their erythrocytes count (see FIG. 1) showed a time-dependant increase from baseline to Week 20 (p=0.008), followed by a steady plateau phase extending to Week 60 showing a 5.4% increase and in treated group (from 4.330 at baseline to 4.582 during plateau) over placebo (from 4.367 at baseline to 4.385 during plateau). A similar trend was also seen in hematocrit. Until Week 20, hematocrit showed a time-dependant increase from baseline (p=0.001), followed by a plateau phase lasting to Week 60 in the shark cartilage treated group (see FIG. 2).

Plateau hematocrit value was 3.9% higher in the treated group (from 38.1% at baseline to 40.2% during plateau; Δ=2.1%) compared to placebo (37.7% at baseline to 38.3% during plateau; Δ=0.6%).

Hemoglobin values showed a similar trend (p=0.10). These variations were seen on the entire cohort of 305 patients and were not influenced by sex, age, number of metastatic sites, or ECOG status of patients.

It is interesting to note that changes of the same amplitude in blood parameters were seen in recombinant human erythropoietin (rHuEPO) clinical trials. Clinical trials with various rHuEPO showed an increase in hematocrit of 2.9% (in absolute value) over placebo in a population of cancer patients with no concomitant chemotherapy (see Table 3 below). Epogen® and Procrit®¹ are two commercially available drugs containing rHuEPO for management of anemia.
Epogen® is a registered trademark of Amgen Inc.; Procrit® is a registered trademark of Johnson & Johnson Corporation.

TABLE 3
Changes in hematocrit induced by rHuEPO in clinical trials:

| | No | | Chemotherapy | | Platinum | |
|---|---|---|---|---|---|---|
| | Chemotherapy | | No platinum | | Chemotherapy | |
| | EPO | Ctrl | EPO | Ctrl | EPO | Ctrl |
| Hematocrit** | 2.8 | −0.1 | 6.9 | 1.1 | 6 | 1.3 |

**Variation from baseline in % of hematocrit value in cancer patients
Source: Ray-Coquard et al.

Over 800 patients with angiogenesis-related conditions have been treated with shark cartilage extract worldwide. The product has proven to be safe and biologically active. Benefits of shark cartilage extract treatment for cancer patients included an increase in median survival time, and an upregulation of blood parameters toward normal values or a significant increase in blood parameters. Maintaining proper blood parameter levels in cancer patients may improve their quality of life and positively influence the course of their illness.

The statistical analysis of the results was conducted by an independent statistician and was aimed at minimizing standard deviation by including all patients, instead of a more conventional approach which would presuppose elimination of any patients with a missing data between weeks 0 and 20

### EXAMPLE 3

### Administration of Shark Cartilage Extract to Healthy Individuals

A randomized, double-blinded, placebo-controlled trial was conducted on healthy human subjects to measure the effect of shark cartilage extract on their blood parameters.

The shark cartilage extract was obtained as described in Example 1 and further diluted with pure water for a final concentration of 71.5% of the original shark cartilage extract. It is to be understood that a quantity of original (undiluted) shark cartilage extract equivalent to the diluted shark cartilage extract described in the present Example could have been provided to the patient, namely 5 ml and 15 ml. Any later reference to “shark cartilage extract” in the present Example is meant to refer to shark cartilage extract obtained as described in Example 1 and further diluted as described above.

Twenty-nine healthy male volunteers were randomized to receive a placebo, the shark cartilage extract (7 mL) or shark cartilage extract (21 ml) through oral administration on a daily basis, from day 1 to day 23 of the study protocol. Blood samples were drawn prior to the first shark cartilage extract administration on day 0 (baseline), at day 11 (middle) and at day 23 (end). These samples were used for determination of blood cell counts (erythrocytes count, hematocrit, haemoglobin). Blood parameters were within normal ranges for both groups at baseline (Table 4)

TABLE 4
Blood parameters of healthy volunteers following supplementation
of placebo or 7 ml of shark cartilage extract

| | | | |
|---|---|---|---|
| Parameter | Day 0 | Day 11 | Day 23 |

Erythrocyte count (cells × 10¹²/l)

| | | | |
|---|---|---|---|
| Placebo | 5.32 ± 0.03 | 5.28 ± 0.05 | 5.29 ± 0.05 |
| shark | 4.85 ± 0.08 | 5.08 ± 0.10 | 5.02 ± 0.06 |
| cartilage | | | |
| extract | | | |
| 7 ml | | | |
| Normal | 4.6-5.4 | | |
| range | | | |
| for man | | | |

Hematocrit (%)

| | | | |
|---|---|---|---|
| Placebo | 45.96 ± 0.53 | 46.20 ± 0.89 | 45.94 ± 0.61 |
| shark | 42.49 ± 0.62 | 44.34 ± 0.74 | 43.43 ± 0.52 |
| cartilage | | | |
| extract | | | |
| 7 ml | | | |
| Normal | 41-47 | | |
| range | | | |
| for man | | | |

Hemoglobin (g/dl)

| | | | |
|---|---|---|---|
| Placebo | 15.64 ± 0.17 | 15.38 ± 0.27 | 15.40 ± 0.15 |
| shark | 14.48 ± 0.27 | 15.07 ± 0.29 | 14.88 ± 0.22 |
| cartilage | | | |
| extract | | | |
| 7 ml | | | |
| Normal | 14.0-16.0 | | |
| range | | | |
| for man | | | |

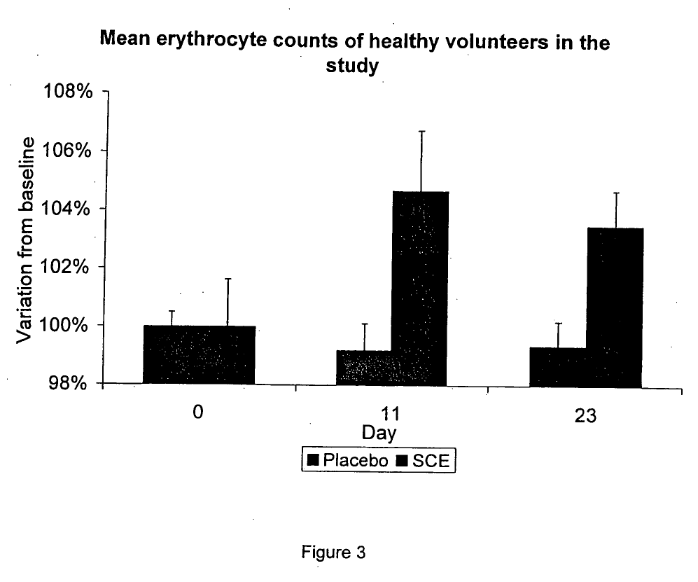

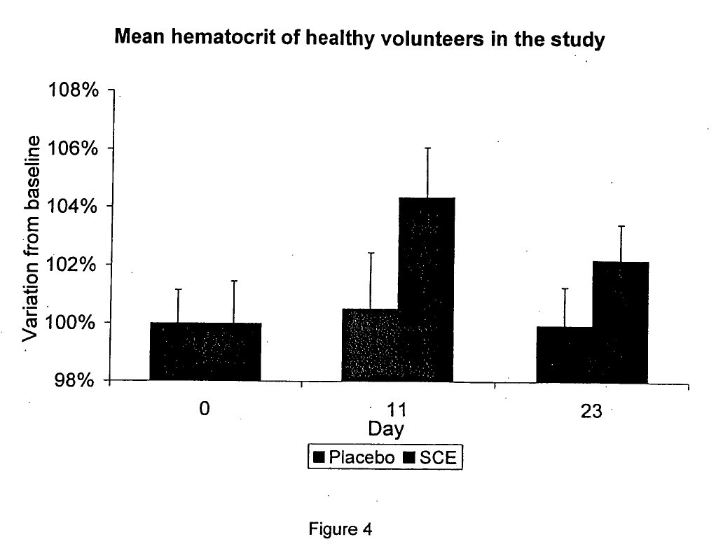

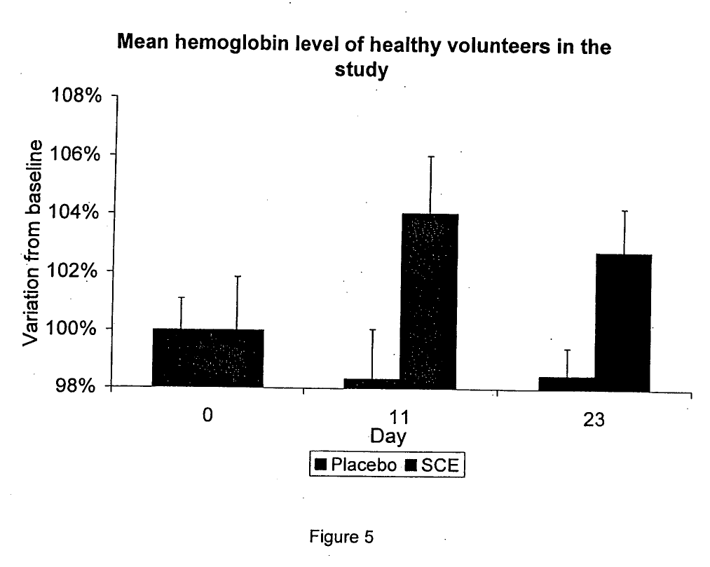

Results from this study evidenced a slight increase at Day 11 in erythrocytes counts (Δ=0.27×10¹² cells/L), hematocrit values (Δ=1.62%), and hemoglobin levels (Δ=0.85 g/dL) for those who received the 7 ml shark cartilage extract over the placebo group (FIGS. 3 to 5). The gains for the shark cartilage extract group were maintained by the end of the study.

There is a direct link between hemoglobin concentrations in blood and the ability to use oxygen, as measured by VO₂max. When hemoglobin concentration is higher, the blood can carry more oxygen. This builds up a functional reserve that pushes forward the limits of the body. Scientific studies have shown that each increment of 1 g/dL in hemoglobin increases physical performances by 1-4% (Warren G L and Cureton K J, 1989) From these data, a 0.85% to 3.4% increase in physical performance with shark cartilage extract can be extrapolated. These results are about the same that can be achieved with altitude training over a 2 to 3 week period, and could be enough for an endurance athlete to win the extra minutes that will put him on the podium.

Although the present invention has been described hereinabove by way of preferred embodiments thereof, it can be modified, without departing from the spirit and nature of the subject invention as defined in the appended claims.

In a preferred embodiment, the invention method of increasing at least one blood parameter in a mammal is preferably used by mammals desirous of the benefits noted herein, subjects “in need of” these benefits. Such subjects are typically looking to adjust erythrocyte cell count/total erythrocyte number in a given volume, hematocrit level, haemoglobin level/concentration, etc. and/or are at recognized and appreciated risk of developing conditions such that they desire to use the invention methods and compositions to combat anticipated effects. Naturally, one using the invention as disclosed will use or administer an amount of the invention composition effective to increase at least one blood parameter in a mammal. Such amounts are inclusive of an amount of the compositions described herein at the disclosed concentrations of active ingredients, and includes repeated administration, for example on a daily basis over a course of days, weeks, etc. Based on the present disclosure one of ordinary skill is able to determine dosage and administration without undue effort. In a preferred embodiment the invention process includes multiple applications of the invention.

### REFERENCES

Béliveau R, Gingras D, Kruger E A, Lamy S, Sirois P, Simard B, Sirois M G, Tranqui L, Baffert F, Beaulieu E, Dimitriadou V, Pepin M C, Courjal F, Ricard I, Poyet P, Falardeau P, Figg W D, Dupont E. The antiangiogenic agent Neovastat (AE-941) inhibits vascular endothelial growth factor-mediated biological effects. Clin Cancer Res. April 2002; 8(4): 1242-50.
Berbari P, Thibodeau A, Germain L, Saint-Cyr M, Gaudreau P, Elkhouri S, Dupont E, Garrel D R, Elkouri S, El-Khouri S. Antiangiogenic effects of the oral administration of liquid cartilage extract in humans. J Surg Res. November 1999; 87(1): 108-13.
Blain E J, Gilbert S J, Wardale R J, Capper S J, Mason D J, Duance V C. Up-regulation of matrix metalloproteinase expression and activation following cyclical compressive loading of articular cartilage in vitro. Arch Biochem Biophys. Dec. 1, 2001; 396(1): 49-55.
Chang C, Werb Z. The many faces of metalloproteases: cell growth, invasion, angiogenesis and metastasis. Trends Cell Biol. November 2001; 11(11): S37-43. Review.
Clinical Laboratory Testings, Ed. McGraw-Hill, 2001.
Costelli P, Carbo N, Tessitore L, Bagby G J, Lopez-Soriano F J, Argiles J M, Baccino F M. Tumor necrosis factor-alpha mediates changes in tissue protein turnover in a rat cancer cachexia model. J Clin Invest. December 1993; 92(6): 2783-9.
Coutts R D, Healey R M, Ostrander R, Sah R L, Goomer R, Amiel D. Matrices for cartilage repair. Clin Orthop. October 2001; (391 Suppl): S271-9. Review.
Dufour C, Corcione A, Svahn J, Haupt R, Poggi V, Beka'ssy A N, Scime R, Pistorio A, Pistoia V. TNF-alpha and IFN-gamma are overexpressed in the bone marrow of Fanconi anemia patients and TNF-alpha suppresses erythropoiesis in vitro. Blood. Sep. 15, 2003; 102(6): 2053-9.
Dunst J, Becker A, Lautenschlager C, Markau S, Becker H, Fischer K, Haensgen G. Anemia and elevated systemic levels of vascular endothelial growth factor (VEGF). Strahlenther Onkol. August 2002; 178(8): 436-41.
Dupont E, Falardeau P, Mousa S A, Dimitriadou V, Pepin M C, Wang T, Alaoui-Jamali M A. Antiangiogenic and antimetastatic properties of Neovastat (AE-941), an orally active extract derived from cartilage tissue. Clin Exp Metastasis. 2002; 19(2): 145-53.
Dupont E, Wang B, Mamelak A J, Howell B G, Shivji G, Zhuang L, Dimitriadou V, Falardeau P, Sauder D N. Modulation of the Contact Hypersensitivity Response by AE-941 (Neovastat), a Novel Antiangiogenic Agent. J Cutan Med Surg. Feb. 10, 2003.
Evans W K, Champagne P, Falardeau P, Hariton C, Dupont É. Neovastat (AE-941) in addition to combined modality therapy for locally-advanced unresectable Non-Small Cell Lung Cancer (NSCLC): a NCI-sponsored multicenter Phase III study. Proc. AACR-NCI-EORTC International Conference held in Miami, (Fla.), Oct. 29-Nov. 2, 2001. Clin Cancer Res 2001: 7 (11):Suppl. (abstract 32).
Fortunel N O, Hatzfeld A, Hatzfeld J A. Transforming growth factor-beta: pleiotropic role in the regulation of hematopoiesis. Blood. Sep. 15, 2000; 96(6): 2022-36. Review.
Gelin J, Moldawer L L, Lonnroth C, Sherry B, Chizzonite R, Lundholm K. Role of endogenous tumor necrosis factor alpha and interleukin 1 for experimental tumor growth and the development of cancer cachexia. Cancer Res. Jan. 1, 1991; 51(1): 415-21.
Gingras D, Renaud A, Mousseau N, Beaulieu E, Kachra Z, Beliveau R. Matrix proteinase inhibition by AE-941, a multifunctional antiangiogenic compound. Anticancer Res. January-February 2001; 21 (1A): 145-55.
Harrison's Principles of Internal Medicine, 14^{th} edition, Ed. McGraw-Hill, 1998.
Imai K, Hiramatsu A, Fukushima D, Pierschbacher M D, Okada Y. Degradation of decorin by matrix metalloproteinases: identification of the cleavage sites, kinetic analyses and transforming growth factor-beta1 release. Biochem J. Mar. 15, 1997; 322 (Pt 3): 809-14.
Janusz M J, Bendele A M, Brown K K, Taiwo Y O, Hsieh L, Heitmeyer S A. Induction of osteoarthritis in the rat by surgical tear of the meniscus: Inhibition of joint damage by a matrix metalloproteinase inhibitor. Osteoarthritis Cartilage. October 2002; 10(10): 785-91.
Ray-Coquard I, Bachelot T, Sebban C, Biron P, Guastalla J P, Blay J Y. Anaemia and cancer. Electronic J. Oncol. 2002 (1): 1-16
Warren G L, Cureton K J. Modeling the effect of alterations in hemoglobin concentration on VO2max. Med Sci Sports Exerc. October 1989; 21(5): 526-31. Review.

What is claimed is:

1. A method of increasing at least one blood parameter in a mammal, comprising administering cartilage extract to said mammal, with the proviso that when said at least one blood parameter is low prior to administering said cartilage extract, said mammal is not co-administered an anticancer agent.

2. A method as in claim 1, wherein the at least one blood parameter is erythrocyte cell count/total erythrocyte number in a given volume.

3. A method as in claim 1, wherein the at least one blood parameter is hematocrit level.

4. A method as in claim 1, wherein the at least one blood parameter is haemoglobin level/concentration.

5. A method as in any one of claim 1, wherein said cartilage extract is shark cartilage extract.

6. A method as in claim 1, wherein said mammal is a human.

7. A method as in claim 1, wherein said at least one blood parameter is low in said mammal prior to administering said cartilage extract.

8. A method as in claim 1, wherein said mammal is a human which has cancer.

9. A method as in claim 8, wherein said cancer is late stage renal cell carcinoma.

10. A method as in claim 1, wherein said at least one blood parameter is normal in said mammal prior to administering said cartilage extract.

11. A method as in claim 2, wherein said cartilage extract is shark cartilage extract.

12. A method as in claim 3, wherein said cartilage extract is shark cartilage extract.

13. A method as in claim 4, wherein said cartilage extract is shark cartilage extract.

14. A method as in claim 2, wherein said mammal is a human.

15. A method as in claim 3, wherein said mammal is a human.

16. A method as in claim 4, wherein said mammal is a human.

17. A method as in claim 5, wherein said mammal is a human.

18. A method as in claim 6, wherein said at least one blood parameter is low in said mammal prior to administering said cartilage extract

19. A pharmaceutical and/or nutraceutical and/or dietary composition comprising a cartilage extract and at least one pharmaceutically acceptable auxiliary, additive and/or carrier.

20. A composition as in claim 19, wherein said cartilage extract is shark cartilage extract.
